(19) United States
(12) Patent Application Publication
(10) Pub. No.: US 2005/0059839 A1
(43) Pub. Date: Mar. 17, 2005

(54) Process and catalysts for the oxidation of methanol and/or ethanol

(75) Inventors: Haichao Liu, Beijing (CN); Enrique Iglesia, Moraga, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 10/663,299

(22) Filed: Sep. 15, 2003

(51) Int. Cl. C07C 69/02; C07C 43/30; C07C 43/32

(52) U.S. Cl. 560/231; 568/594

(57) ABSTRACT

A process for oxidation of methanol, ethanol, or a mixture of methanol and ethanol comprising contacting the methanol and/or ethanol with an oxygen-containing gas and a supported catalyst comprising one or more platinum group oxides. The process conditions and/or catalyst may be selected to as to selectively produce methyl formate from methanol or diethoxyethane from ethanol. The invention also includes certain novel supported platinum group metal oxide catalysts. Preferred catalysts include one or more ruthenium oxides.

### BACKGROUND AND FIELD OF THE INVENTION

In its broadest sense this application relates to a process (and in a preferred embodiment, to a novel catalyst) for the oxidation of methanol or ethanol, or a mixture of methanol and ethanol, comprising contacting the methanol and/or ethanol with an oxygen-containing gas and a supported catalyst that comprises one or more platinum group metal oxides. In the case of methanol, the products may include methyl formate, formaldehyde and methylal (dimethoxymethane). In a preferred embodiment, the process is conducted so as to be relatively selective for production of one or more particular products, for example primarily methyl formate or mixtures of methyl formate and dimethoxymethane, from the methanol. At the same time, production of undesired combustion products such as carbon oxides, as well as dimethyl ether, can be inhibited. Production of dimethyl ether in such a process is less desirable as it may revert to methanol. These objectives may be achieved by employing preferred novel catalysts of this invention and/or by controlling the reaction conditions. Use of the preferred novel catalysts can result in selectivity to methyl form ate at lower temperatures as compared to prior art catalysts. If ethanol is oxidized using this invention, the primary products tend to be 1,1-diethoxyethane (DEE) and acetaldehyde, with little ester production.

Methyl formate is a significant industrial product, used for example as an intermediate in the production of formic acid, dimethylformamide (DMF), and acetic acid. Typically this compound is produced either by catalytic non-oxidative dehydrogenation of methanol or by reaction of methanol with carbon monoxide in the presence of a catalyst. Diethoxyethane is used as solvent and as an electrolyte, and finds particular use as a component of fuel cells and as an oxygenated fuel additive. It usually is produced by an acid-catalyzed acetalization reaction of acetaldehyde and ethanol.

Production of methyl formate by catalytic non-oxidative dehydrogenation of methanol has been described in a number of patents and articles, and using a variety of catalysts. Such processes typically result in production of dimethoxymethane, formaldehyde, and dimethyl ether, in addition to methyl formate. Temperatures, for the most part, run from about 180 to about 350° C.

For instance, U.S. Pat. No. 4,149,009 (Yoneoka et al.) describes such a process in which the catalyst contained copper, zirconium and zinc, and optionally aluminum, and in which a nonreactive gas such as hydrogen, carbon monoxide or nitrogen is employed. This process does not use oxygen as a co-reactant. The examples show operation at temperatures of from 230 to 330° C. Selectivity to methyl formate increased with increasing reaction time, often reaching above 90% after 50 hours or more. In U.S. Pat. No. 4,232,171 (also of Yoneoka et al.) in a similar process, the catalyst contained a copper compound and cement. Temperatures were from 192 to 260° C. with selectivities to methyl formate reaching above 90% in one example. U.S. Pat. No. 4,319,037 (Yoneoka) describes such a non-oxidative process using a catalyst containing a copper compound and a compound of a Group IIIA or IVA metal. Temperatures ranged from 139 to 301° C. Methyl formate selectivities of up to 97% were achieved, though often the selectivity was accompanied by a low overall conversion of methanol.

U.S. Pat. No. 4,480,122 (Horlenko et al.) describes a process for non-oxidative dehydrogenation of methanol using copper-containing catalysts supported on spinels. Temperatures here were at higher levels of from 222 to 330° C. Selectivity to methyl formate was as low as 9% and as high as 91% with variations in the reaction conditions. In U.S. Pat. No. 4,778,923 (Aplin et al.) the catalyst in another such process was a platinum group metal, preferably a ruthenium-phosphine complex, and temperatures ranged from 148 to 180° C. Yields of methyl formate, however, were quite low, with hydrogen being the major reaction product. In U.S. Pat. No. 5,144,062 (Chen et al.) the catalysts for this reaction contained a copper oxide, a chromium oxide, and a sodium compound such as sodium oxide, hydroxide, carbonate or bicarbonate. Temperatures ranged from 140 to 180° C. Selectivity to methyl formate was high, ranging from 81.2 to as high as 97.8%. However, the yield of methyl formate in this process is generally below 50%, limited by the thermodynamics of the reaction.

U.S. Pat. No. 5,194,675 (Joerg et al.) discloses a non-oxidative dehydrogenation process in which the catalyst contained copper supported on magnesium silicate, optionally doped with one or more other metals. Process temperatures ranged from 160 to 260° C., with selectivity to methyl formate ranging from 48.1 to 98.0%. In U.S. Pat. No. 5,399,745 (Yoneoka et al.) the reaction was run in the liquid rather than gas phase at 195-199° C. using a copper-zinc-aluminum-containing catalyst. Methyl formate selectivities ranged from 74.8 to 92.5%.

Processes for production of methyl formate by reaction of methanol with carbon monoxide (i.e., carbonylation) are described, for instance, in U.S. Pat. No. 4,216,339 (Couteau et al.), U.S. Pat. No. 4,994,603 (Mueller et al.), U.S. Pat. No. 5,401,873 (Zehner et al.), and U.S. Pat. No. 5,917,085 (Lippert et al.). U.S. Pat. No. 5,026,904 (Lodge et al.) discloses production of formates by reaction of hydrogen, carbon dioxide and an alcohol. U.S. Pat. No. 5,223,102 (Fedkiw, Jr., et al.) discloses a process for electrooxidation of methanol to produce formaldehyde and methylal.

Production of methyl formate by oxidation of methanol is described in the technical literature. For example, Tronconi et al., Ind. Eng. Chem. Res. 1987, 26:1269 described such a reaction using a vanadium/titanium oxide catalyst with about 80% selectivity to methyl formate at 170° C. (443 K). In Ai, J. Catal. 1982, 77:279 the catalyst was a molybdenum/stannic oxide combination. Methyl formate selectivity was 90% at 160° C. (433 K). Valente et al., Appl. Catal. 2001, 205:201 also used a molybdenum/stannic oxide catalyst, at 180° C. (453 K). However, selectivity to methyl formate was low (47.0%) and a substantial conversion of methanol to dimethyl ether occurred (selectivity—24.2%).

The literature also contains a number of publications describing the use of platinum-group metal catalysts for oxidation of alcohols to produce aldehydes and/or ketones. These, however, do not relate to oxidation of methanol or ethanol, but to oxidation of higher alcohols such as longer-chain saturated or unsaturated aliphatic alcohols or aromatic or other cyclical compounds including benzyl and cinnamyl alcohols. Results of such work are described, for instance, by Stuchinskaya et al., Catalysis Communications 2003, 4:417. In a few instances, alcohols containing as few as four or five carbon atoms were included in the experiments; however, again the products were aldehydes and ketones (Matsumoto et al., J. Org. Chem. 1984, 49:3435; Yamaguchi et al., Angew. Chem. Int. Ed. 2002, 41:4538; Zhan et al., JACS 2003, 125:2195).

Oxidation of ethanol to acetic acid is described, for example in U.S. Pat. No. 5,770,761 (Lin et al.) and U.S. Pat. No. 5,840,971 (Gubelmann-Bonneau).

Previous work on conversion of methanol to methyl formate appears to have focused primarily on non-oxidative dehydrogenation, which is relatively disfavored thermodynamically. Work on oxidation of alcohols appears to have focused on higher alcohols, which are relatively easy to dehydrogenate oxidatively to form carbonyl compounds. It thus would be desirable to provide a process for oxidation of methanol that takes advantage of the relatively favorable thermodynamics of an oxidation process. It would be desirable to provide a process for production of methyl formate from methanol with high selectivity for methyl formate but at lower reaction temperatures than used in the above-described processes. It would also be desirable to provide such a process that did not result in significant quantities of dimethyl ether. It also would be desirable to provide a process for oxidation of ethanol to diethoxyethane.

### BRIEF SUMMARY OF THE INVENTION

In one aspect, this invention comprises a process for oxidation of methanol, ethanol, or a mixture of methanol and ethanol comprising contacting the methanol and/or ethanol with an oxygen-containing gas and a catalyst, preferably a supported catalyst, comprising one or more oxides of a platinum group metal, preferably ruthenium oxide. Preferred catalyst supports comprise alumina, zirconia, stannic oxide, titania, silica, ferric oxide, ceric oxide, other acidic or reducible metal oxides, and mixtures and combinations thereof. In one preferred embodiment methanol is oxidized with good selectivity to methyl formate. In another preferred embodiment this invention comprises such a process in which the ruthenium and/or other platinum group metal oxides are dispersed on the surface of the support, and in which the surface density of the oxide on the support is greater than that for the isolated monomeric oxide. Most preferably the surface density of the platinum group metal oxide(s) on the support is approximately that of a monolayer of the oxide at the surface of the support. Preferably such a catalyst would contain little or no bulk crystalline platinum group metal oxides.

However, in its broadest sense the invention comprises a process for the oxidation of methanol and/or ethanol comprising contacting the methanol and/or ethanol with an oxygen-containing gas and a supported catalyst that comprises one or more platinum group metal oxides, and the processes of the invention can be carried out using commercially available or otherwise known supported platinum group metal oxide catalysts.

In another preferred embodiment, the catalyst comprises one or more reducible metal oxides. More preferably in this embodiment, the catalyst comprises a layer of the reducible metal oxide or oxides, most preferably stannic oxide, on a particulate support (preferably alumina and/or zirconia) with platinum group metal oxide or oxides being present as an upper layer or layers on the layer of reducible metal oxide(s). In this embodiment, preferably the surface density of the platinum group metal oxide or oxides on the support is greater than that for the isolated monomeric oxide or oxides. Most preferably the surface density of the platinum group metal oxide on the support is approximately that of a monolayer of oxide at the surface of the support.

Catalysts of the above type in which the catalyst comprises one or more reducible metal oxides, particularly stannic oxide, and more particularly in which the platinum group metal oxide is supported on a layer or layers of reducible metal oxide or oxides, with the oxide layer or layers being disposed on a particulate support comprising alumina and/or zirconia and/or silica, are novel and form another feature of this invention.

The invention further comprises a process for the production of methyl formate, dimethoxymethane, formaldehyde, or mixtures of two or all three of them, comprising contacting methanol with an oxygen-containing gas and a catalyst comprising one or more platinum group metal oxides. In another aspect it comprises a process for producing diethoxyethane comprising contacting ethanol with an oxygen-containing gas and a catalyst comprising one or more platinum group metal oxides. In yet another aspect, it comprises a process for oxidizing a mixture of methanol and ethanol with an oxygen-containing gas and a catalyst comprising one or more platinum group metal oxides.

### DETAILED DESCRIPTION OF THE INVENTION

As used herein, terms have the following meanings:
“Platinum group metal” includes platinum, palladium, ruthenium, rhodium, osmium and iridium.
“Platinum group metal oxide/oxides” means one or more oxides (as indicated) of one or more platinum group metals. As is known, members of this group of metals generally have several valences which may range from +2 to +8. A catalyst containing such oxides may contain a single oxide of a platinum group metal wherein the metal has a single valence, a mixture of oxides of such a metal in which the metal has different valences, or oxides of two or more platinum group metals in which each of the metals may have more than one valence. For convenience, the term “platinum group metal oxide” will often be used. However, unless the context clearly indicates otherwise, this term is meant to include a mixture of two or more such oxides as well as a single oxide.
Ruthenium oxide” or “RuOₓ” similarly means one or more oxides of ruthenium. Ruthenium may have a valence of +2, 3, 4, 6 or 8, and thus may form a number of oxides. The most stable ruthenium oxide is ruthenium dioxide, RuO₂, and the most preferred ruthenium oxide catalysts of this invention will have a ruthenium:oxygen ratio of approximately 1:2. In a given catalyst containing ruthenium oxide, however, the ruthenium may have multiple valences and the ruthenium:oxygen ratio may vary.

In one aspect this invention comprises a process for production of a product comprising primarily methyl formate from methanol comprising contacting the methanol with an oxygen-containing gas and a supported catalyst comprising one or more platinum group metal oxides, preferably ruthenium oxide. As will be described below, preferably the catalyst further comprises a support comprising silica, alumina, zirconia, titania and/or stannic oxide. Some of the catalysts used for this process are known in general, although for other processes. However, this invention also comprises novel supported catalysts comprising platinum group metal oxides, preferably ruthenium oxide.

In another aspect this invention generally comprises a process for oxidation of methanol or ethanol, or a mixture of methanol and ethanol, comprising contacting the methanol or ethanol, or the mixture, with an oxygen-containing gas and a supported catalyst that comprises one or more platinum group metal oxides. Mixtures of methanol and ethanol may comprise primarily one of them, with the other present in a minor amount (for example as a byproduct or impurity) or they may contain substantial amounts of both compounds. In another aspect the invention comprises a process for producing diethoxyethane from ethanol comprising contacting the ethanol with an oxygen-containing gas and a supported catalyst that comprises one or more platinum group metal oxides. In other aspects the invention comprises novel platinum group metal containing-oxide catalysts as described herein.

In the processes of this invention a feed comprising methanol and/or ethanol is contacted with a supported catalyst comprising one or more platinum group metal oxides, preferably ruthenium oxide. The reaction temperature can range from about 30 to about 300° C., preferably from about 50 to about 180° C., and most preferably from about 80 to about 180° C. The pressure is from about 0.5 to about 2000, preferably from about 2 to about 500 psig; the reaction time is from about 0.5 to about 1000, preferably from about 1 to about 500 hours. Space velocity is from about 30 to about 50,000 hr⁻¹, preferably from about 100 to about 20,000 hr⁻¹. Methanol or ethanol concentration in the flow is from about 0.5 to about 99%, preferably from about 2 to about 90%. In one embodiment the process is a gas phase process.

As will be seen from the examples below, variation in process conditions can result in differences in conversion or selectivity. In general, for methanol as a feed, with increasing temperature conversion increases while selectivity to methyl formate decreases, with concomitant increase in the production of carbon oxides. Selectivity to methyl formate tends to be highest at lower temperatures. In the oxidation of ethanol, selectivity to diethoxyethane is greater as the partial pressure of ethanol in the feed increases. At lower partial pressures of ethanol acetaldehyde tends to be the primary product.

The process for oxidation of methanol or ethanol or their mixtures involves contacting that substance or substances with an oxygen-containing gas in the presence of a supported platinum group metal catalyst. The oxygen-containing gas used in the process may be in the form of molecular oxygen, a commercial mixture of molecular oxygen with an inert gas such as nitrogen, air, or oxygen-enriched air, but is preferably substantially pure oxygen or a commercial mixture that contains predominantly oxygen. The ratio of oxygen (calculated as O₂) to alcohol is from about 50 to about 0.05, preferably from about 20 to bout 0.2.

In practice, the processes of this invention may be run in equipment ranging in size from microreactors (e.g. microchannel reactors) to full-sized commercial process equipment. A commercial installation will include typical process expedients such as recycle streams, for efficient use of reactants and reaction products, and may be integrated with process units for production of products from the methyl formate and/or other products of the reaction. For example, if the process is aimed at producing methyl formate from methanol, dimethoxymethane in the reaction products may be recycled for production of additional methyl formate.

The catalysts used in the processes of this invention comprise one or more platinum group metal oxides. A preferred oxide is ruthenium oxide, which is generally indicated herein as RuOₓ, in which the ruthenium may be present at a valence of from +2 to +8. The catalysts contain from about 0.1 to about 80, preferably from about 0.5 to about 20, weight % ruthenium or other platinum group metal oxide. The platinum group metal oxide (or oxides) is supported on a particulate support comprising alumina (Al₂O₃), zirconia (ZrO₂), silica (SiO₂), titania (TiO₂) or stannic oxides (SnO₂), or a mixture of two or more of these. The catalyst may be a conventional supported platinum group metal oxide catalyst, but more preferably is a novel catalyst of this invention. In one preferred embodiment of the invention the platinum group metal oxide is dispersed on the surface of the support, and the surface density of the oxide on the support is greater than that for the isolated monomeric oxide or oxides. In one embodiment of these novel catalysts the support further comprises one or more reducible metal oxides, and in a preferred embodiment of this type of catalyst the platinum group metal oxide is dispersed on a layer or layers of a reducible metal oxide or oxides that is further supported on alumina, titania, silica, stannic oxide or zirconia (if stannic oxide or zirconia is not used as the reducible metal oxide layer).

In the catalysts of this invention, the platinum group metal oxide, e.g., ruthenium oxide, is distributed on the surface of the support material in what is generally denoted as a distribution of small domains, essentially amorphous, substantially without sharp X-ray diffraction lines. The surface density of the oxide catalyst on the support (measured in units of metal atoms per nm²) is chosen so as to be greater than the surface density of the respective isolated monomeric oxide or oxides, but well below the surface density that would characterize multiple layers of the oxide(s). However, the catalyst overall is preferably characterized by a substantial absence of bulk crystalline oxides. By “bulk crystalline oxides” is meant oxide(s) having a clear X-ray diffraction pattern. The crystallinity can be determined by X-ray diffraction based on the peak intensity ratio between one of the peaks of the supported metal oxide and one of the peaks of the support employed after calibration with a mixture of known amounts of the metal oxide and the support. By “substantial absence” is meant that the supported catalyst contains less than about 5% of the bulk crystalline ruthenium or other platinum group metal oxide.

Surface densities of the catalysts described herein are given in terms of nominal surface density. This value is calculated based on the elemental analysis of the platinum group metal oxide and on the surface area of the support, i.e., by dividing the number of platinum group metal atoms in a given mass of sample by the surface area of the support (calculated from N₂ absorption at its normal boiling point using the Brunauer-Emmett-Teller, or BET, equation).

The surface density of the catalyst can affect the catalyst efficiency. At one extreme, catalysts of this type with relatively isolated oxide species have relatively few active sites on the support surface. These catalysts tend to retain their oxygen and thus provide lower reaction rates for the oxidation of methanol to methyl formate or of ethanol to diethoxyethane per exposed active species. At the other extreme, catalysts having a rather high density of platinum group metal oxide (e.g., bulk crystals) may possibly provide reasonable reaction rates per unit surface area. However, they also lack efficiency in the utilization of the oxide catalyst because a substantial amount of the oxide is located within the crystals and is thus not available for catalyzing the reaction. Bulk crystals also tend to be nonselective in their functioning, and can cause overreaction to produce carbon oxides rather than methyl formate and other desired products.

It has been found that the most preferred catalysts for this reaction tend to have a surface density of approximately a monolayer of catalyst on the support. For ruthenium oxide-containing catalysts the monolayer surface density is approximately 6 Ru/nm². The terms “monolayer” and “monolayer capacity value” as used herein are meant to refer to this value. A preferred range of surface densities is from about 20 to about 300% of the monolayer capacity value for all supports usable in the catalysts of this invention.

When ruthenium oxide is used, it may be present as the oxide per se, represented by the general formula RuOₓ, where x represents general values for oxygen in such molecules. This oxide generally comprises about two oxygen atoms per ruthenium atom; i.e., the general form of the oxide may be represented as RuO₂, or ruthenium dioxide. However, in a given case the oxide may have an oxygen-to-metal atomic ratio that is not necessarily exactly 2:1. Likewise, oxides used as a component of the support may be represented by more general formulas such as SnOₓ, FeOₓ and CeOₓ where the oxides generally comprise about 2, 1.5 and 2 oxygen atoms per metallic atom, respectively. However, in a given case, such oxide similarly may have an oxygen-to-metal atomic ratio that is not exactly these values.

In addition, the platinum group metal oxide, such as ruthenium oxide, may form one or more complexes or compounds with the support. These complexes usually also will be an oxide. In any case, such complexes formed with ruthenium oxide or other platinum group metal oxides are considered to be within the definition of the oxide catalysts to which this invention pertains.

The support may be selected among commonly used supports for such oxide catalysts, including mixtures of such supports. The properties of the support will influence both the acid and redox properties of the overall catalyst, and consequently the product distribution in methanol and ethanol oxidation. On acidic and refinery supports (such as alumina, silica, and mixtures of them, dimethoxymethane is the main product of methanol oxidation, whereas methyl formate is the primary product when supports containing amphoteric and reducible metal oxides that are known to catalyze hydrogenation-dehydrogenation reactions are used. Some properties may make certain supports unsuitable for use in the process of this invention. For instance, supports that will react with the platinum group metal oxide to form any significant amounts of unreducible mixed bulk oxides, i.e. oxides that would not undergo substantial formation of oxygen vacancies at temperatures below about 250° C., would in general not be suitable for use in this process. One commonly used catalyst support, magnesium oxide, for example, was tested for suitability and was found unsuitable as a support for ruthenium oxide in these oxidation processes, perhaps for this reason. Supports that could cause undesired combustion of products to form carbon oxides under the operating conditions of this process also would not be suitable for use in this invention. In one preferred embodiment of the invention, the supports should allow or favor the formation of a monolayer of platinum group metal oxide on the surface of the support.

Preferred supports include acidic oxides such as alumina and silica, acid-basic oxides such as Mg—Al hydrotalcites, and reducible metal oxides such as zirconia, titania, stannic oxide, ferric oxide, ceric oxide, and mixtures or other combinations of two or more of these oxides. Particularly preferred are alumina, silica, zirconia, titania and stannic oxide, and mixtures or other combinations of two or more of them. Most preferred is a catalyst comprising alumina, titania, zirconia or silica modified by the incorporation of a layer or layers of a reducible oxide such as zirconia (if zirconia is not used as the particulate support), stannic oxide, ferric oxide, ceric oxide, or alternatively by incorporation of an (Mg—Al) hydrotalcite (which is not a reducible oxide) deposited thereon. The supports that are suitable for use in this process may be used in any of their available forms, including forms that as of the present time might not yet have been developed, or may have been developed but have not yet been commercialized. Both high and low surface area supports may be used, including materials known by the acronym MCM (standing for “Mobil Compositions of Matter”), e.g., MCM-41. These are recently developed mesoporous materials (often comprising silica) and are described by Kresge, et al. (Nature, 359, 710 (1992)) and by Corma (Chem. Rev., 97, 2373 (1997)). High surface area supports of various physical types are preferred for use in the invention from the point of view of efficiency in that they may produce greater amounts of product per unit mass of overall catalyst.

Reducible metal oxides suitable for inclusion in the catalysts of this invention are those in which at least a fraction of the metal cations undergo a one- or multiple-electron reduction during contact with a reactant such as hydrogen, dimethyl ether, methanol, alkanes or alkenes at typical temperatures of catalytic oxidation reactions, whether or not such metal oxides function as catalyst for the reaction in question. Such reducible metal oxides include reducible oxides of tin, iron, cerium, manganese, cobalt, nickel, chromium, rhenium, titanium, molybdenum, vanadium, silver and copper, and mixtures thereof. Of these, oxides of tin (e.g., stannic oxide), iron (e.g., ferric oxide) and cerium (e.g., ceric oxide) are preferred, with stannic oxide being most preferred for such catalysts of this invention.

Novel catalysts of this invention include those in which the support comprises a layer of a reducible metal oxide disposed on a particulate alumina and/or zirconia (except where zirconia is used as the reducible metal oxide layer), or a layer of zirconia disposed on a particulate alumina, particularly those in which the layer of zirconia has a surface density close to that of a monolayer of that substance. Exemplary catalysts may comprise ruthenium oxide on a near-monolayer of stannic oxide disposed on a particulate (preferably high surface area) alumina or silica. Novel catalysts of this invention also include those in which the reducible metal oxide or oxides are incorporated into the support.

Without intending to be bound by an explanation, it is believed that the reducible metal oxides aid in catalyst performance by decreasing the temperature required for the reduction of some of the platinum group metal oxides. This reduction, in turn, accompanies the use of lattice oxygen atoms in the activation of C—H bonds in methanol and ethanol and their reactive intermediates.

The novel platinum group metal oxide catalysts of this invention that contain reducible metal oxides also are suitable as catalysts for other reactions and processes, for example the oxidation of higher alcohols to the corresponding aldehydes or ketones, or of amines to the corresponding nitrites and imines, and of CO to CO₂ at relatively low temperatures. See, for instance, Yamaguchi et al., Angew. Chem. Int. Ed. 2002, 41, 4538; Zhan, et al, J. Am. Chem. Soc. 2003, 125, 2195; Yamaguchi et al. Angew. Chem. Int. Ed. 2003, 42, 1480; L. Zang et al., Angew. Chem. Int. Ed. 2000, 39, 3921.

The platinum group metal oxide catalysts are prepared by typical means, for instance by impregnation, particularly incipient wetness impregnation, of the support with an aqueous solution containing ruthenium, e.g. using a salt such as RuNO(NO₃)₃, RuCl₃, Ru(NH₃)₆Cl₃, or analogous salts of other platinum group metals such as Pd(NO₃)₂, PdCl₂, Rh(NO₃)₃ and RhCl₃. In a preferred embodiment the preparation is carried out so as to disperse the platinum group metal oxide over the surface of the support, and the amounts are chosen so as to achieve a desired surface density, as described above. Where the catalyst also comprises a reducible metal oxide, for instance as a layer on a particulate support, the reducible metal oxide may be first deposited on the particulate support, for instance by impregnation such as incipient wetness impregnation. Then the platinum group metal oxide is deposited on the support in a second step, e.g. a second impregnation.

Catalysts of this invention may alternatively be prepared by other means such as chemical vapor deposition of layers, precipitation, sol-gel methods, precipitation by controlled addition of a base to a solution of platinum group metal and support cations, chemical anchoring from solution, or ion exchange. Reducible metal oxides may be incorporated into the catalysts either before or after the incorporation of the platinum group metal oxide.

### EXAMPLES

The following are presented as illustrative of the processes and catalysts of this invention. However they are provided by way of illustration only, and not for purposes of limitation of the invention. Those skilled in the art will readily recognize a variety of noncritical parameters that could be changes or modified to yield essentially similar results.

Catalyst Preparation

Supported RuO₂ catalysts were prepared by incipient wetness impregnation of ZrO(OH), Sn(OH)ₓ, TiO₂ (Degussa P25), Al₂O₃ (Alcoa, HiQ31®) and SiO₂ (Cab-O-Sil®) with aqueous solutions of Ru(NO)(NO₃)₃.xH₂O (Alfa Aesar, Ru 32.9 wt %) at 298 K for 5 h, followed by drying in ambient air at 398 K overnight and by treating in dry air flow at 673 K for 2 h. ZrO(OH)₂ was prepared by hydrolysis of aqueous zirconyl chloride solutions (>98%, Aldrich) using NH₄OH (14.8 N), followed by drying in ambient air at 393 K overnight. Sn(OH)ₓ was prepared by hydrolysis of tin (IV) chloride pentahydrate (98%, Alfa Aesar) with NH₄OH (14.8 N), followed by drying in ambient air overnight at 398 K for 3 h.

Note that RuO₃ is known to be volatile at temperatures well above those used in the methanol and ethanol oxidation processes. Production of the catalysts described herein involved a thermal treatment in air at 673 K for 2 h before reaction without detectable loss of Ru oxides. These materials therefore should be able to withstand similar oxidative thermal treatments that may be used in catalyst regeneration.

Processes

Tables 1-5 below contain the results of conducting processes for oxidation of methanol to produce a product containing methyl formate, dimethoxymethane and formaldehyde. Methanol conversion rates and methyl formate synthesis rates were high at temperatures well below those previously reported using other metal oxide catalysts. Some of these prior results are included below for comparison. Table 6 contains results of ethanol oxidation processes; Table 7 contains results of an oxidation process using a mixed methanol/ethanol feed.

In general, the procedure and conditions were as follows:
Methanol reactions were carried out in a fixed-bed quartz microreactor. Catalyst powders (0.1-0.3 g) diluted with acid-washed quartz powder (˜0.5 g) were treated in flowing 20% O₂/He (O₂, Praxair, 99.999%; He, Airgas, 99.999%; 0.67 cm³/s) at 573 K for 1.0 h before catalytic reaction measurements. The reactant mixture consisted of 4 kPa CH₃OH (Merck, 99.99%), 9 kPa O₂, 1 kPa N₂ (Praxair, Certified O₂/N₂ mixture) and 86 kPa balance He (Airgas, 99.999%). Reactants and products were analyzed by on-line gas chromatography (Hewlett-Packard 6890GC) using a methyl-silicone capillary column and a Porapak Q packed column connected to flame ionization and thermal conductivity detectors, respectively. Oxidation of ethanol and of mixtures were carried out similarly, with particular features described below.

Table 1 shows results of catalytic oxidation of methanol on RuOₓ domains supported on SiO₂, Al₂O₃, ZrO₂, TiO₂, SnO₂ and MgO (˜4 wt %) at 393 K (120° C.). Abbreviations used are as follows: CH₃OH—methanol; HCHO—formaldehyde; MF—methyl formate; DMM—dimethoxymethane; COₓ—carbon oxides; RuOₓ—ruthenium oxide (as defined above). All the products just mentioned were formed. However, no dimethyl ether (DME) was detected using any of these supported RuOₓ catalysts at this temperature. CH₃OH conversion rates were higher on SnO₂ and ZrO₂ than on Al₂O₃, TiO₂ and SiO₂. On MgO, only very low CH₃OH conversion was detected. RuOₓ/SnO₂ showed the highest CH₃OH conversion rate, catalyzing CH₃OH conversion to MF, HCHO and DMM with total selectivity of 100% (at ˜10% CH₃OH conversion) at temperatures as low as 333 K (60° C.). On Al₂O₃- and SiO₂-supported RuOₓ samples, 50.1% and 56.1% DMM and 34.4% and 31.0% MF were obtained, respectively; on SnO₂, ZrO₂, and TiO₂, MF was formed at 373 K with selectivities as high as 60-70% and low DMM selectivities (5.5-15.5%) at conversions of ˜20%. This table also includes results reported in the prior art, for comparison. The MF synthesis rates (per active metal site) at 393 K on RuOₓ/ZrO₂, RuOₓ/TiO₂, RuOₓ/SnO₂ are significantly higher than those reported on V—Ti and Mo—Sn oxide catalysts at temperatures above 423 K; the rates per gram catalyst are also higher than those on V—Ti and Mo—Sn oxide catalysts (Table 1).

TABLE 1
Methanol oxidation on RuOₓ supported on SiO₂, Al₂O₃ and ZrO₂, at similar
methanol conversion (˜20%) at 393 K (Ru wt %, ˜4%; 4 kPa CH₃OH, 10 kPa O₂)

| | Conversion | Conversion | Selectivity | | | | |
|---|---|---|---|---|---|---|---|
| | rate | rate | (%) | | | | |
| Catalyst | (mmol/g-Ru-h) | (mmol/g-cat-h) | DME | HCHO | MF | DMM | COₓ |
| RuOₓ/SiO₂ | 82.2 | 3.5 | 0 | 12.4 | 31.0 | 56.1 | 0.6 |
| RuOₓ/Al₂O₃ | 147.9 | 6.4 | 0 | 13.5 | 34.4 | 50.1 | 2.0 |
| RuOₓ/ZrO₂ | 174.5 | 7.3 | 0 | 6.5 | 69.6 | 5.5 | 18.5 |
| RuOₓ/TiO₂ | 108.1 | 4.6 | 0 | 24.9 | 65.2 | 8.9 | 1.0 |
| RuOₓ/SnO₂ | 303.3 | 12.5 | 0 | 20.0 | 60.7 | 15.5 | 3.8 |
| RuOₓ/SnO₂ | 19.4 (333 K) | 0.8 | 0 | 10.2 | 83.1 | 6.7 | 0 |
| RuOₓ/MgO | 2.5 | 0.1 | — | — | — | — | — |
| ^{a}V—Ti—O | ˜66 (443 K) | 2.5 | — | ˜7 | ˜80 | ˜3 | ˜5 |
| ^{b}Mo—Sn—O | ˜10.2 (433 K) | 2.3 | — | 5 | 90 | 0 | 2 |
| ^{c}Mo—Sn—O | ˜18.3 (453 K) | 4.1 | 24.2 | 27.2 | 47.0 | 0 | 1.5 |
| ^{d}MoO₃/SiO₂ | 11.5 (533 K) | — | 1.1 | — | 95.9 | — | 3.0 |

^{a}Tronconi, E., et al, Ind. Eng. Chem. Res., 1987, 26, 1269.
^{b}Ai, M., J. Catal., 1982, 77, 279.
^{c}Valente, N. G., Appl. Catal. A, 2001, 205, 201.
^{d}Louis, C., J. Catal., 1988, 109, 354.

Tests of the supports SiO₂, Al₂O₃, ZrO₂, TiO₂ and SnO₂ alone did not show detectable conversion of methanol to any products at these reaction conditions, even at temperatures up to 493 K (220° C.).

In general, CH₃OH appears to convert to MF and DMM via an initial oxidation to HCHO, followed by subsequent formation of a common methoxymethanol (CH₃OCH₂OH) or hemiacetal and conversion to form MF via dehydrogenation or DMM via a condensation reaction with CH₃OH. More reducible oxides or those that are known to catalyze dehydrogenation reactions (SnO₂, ZrO₂ and TiO₂) favor dehydrogenation steps to form MF, while acidic oxide supports (e.g. Al₂O₃ and SiO₂) preferentially lead to condensation reactions to form DMM.

A parallel study showed that the catalytic properties of RuOₓ depend on size and dispersion, which were varied by changing the Ru surface density on TiO₂. These data are shown in Table 2. As Ru surface density decreased from 13.1 to 0.8 Ru/nm², CH₃OH conversion rates (per Ru-atom) monotonically increased from 69.4 to 265.7 mmol/g-Ru-h at 393 K. This four-fold increase in rate may reflect an increase in the fraction of the RuOₓ species that are exposed to reactants, as low surface densities lead to smaller RuOₓ domains. The selectivity to MF increased from 64.9% to 72.0% (compared at similar CH₃OH conversions) with decreasing Ru surface density (0.8-13.1 Ru/nm²), a trend that leads to a larger fraction of the exposed surface being TiO₂, which appears to be involved in directing reaction intermediates towards dehydrogenation pathways leading to MF. For comparison the catalysts included bulk RuO₂, on which COX selectivity reached values much higher (12.7%) than on RuOₓ/TiO₂ (0-1.4%).

TABLE 2
Surface density effects on methanol oxidation on RuOₓ/TiO₂
(393 K; 0.6-8.2 wt Ru %; 4 kPa CH₃OH, 10 kPa O₂) and
on bulk RuO₂ for comparison at similar CH₃OH conversions
(˜15%)

| Surface density | Conversion rate | Selectivity (%) | | | | |
|---|---|---|---|---|---|---|
| (Ru/nm²) | (mmol/g-Ru-h) | DME | HCHO | MF | DMM | COₓ |
| 0.8 | 265.7 | 0 | 23.2 | 72.0 | 3.9 | 0 |
| 1.6 | 242.4 | 0 | 25.0 | 69.8 | 3.8 | 1.4 |
| 3.1 | 143.2 | 0 | 25.2 | 69.9 | 4.1 | 0.9 |
| 6.2 | 108.1 | 0 | 24.9 | 65.2 | 8.9 | 1.0 |
| 13.1 | 69.4 | 0 | 23.3 | 64.9 | 10.3 | 1.4 |
| Bulk RuO₂ | 24.3 | 0 | 22.5 | 62.1 | 2.7 | 12.7 |

MF selectivities increased slightly and CH₃OH conversion rates remained essentially constant as CH₃OH conversion increased with decreasing reactant space velocity on Ru/ZrO₂ samples. HCHO and DMM selectivities decreased and COX selectivities increased with decreasing space velocity.

Another study showed that the methanol conversion rates increased with increasing reaction temperature between 353 and 408 K (80 and 135° C. respectively) on RuOₓ/ZrO₂. Results are reported in Table 3. MF selectivities decreased and COX concurrently increased with increasing temperature. HCHO and DMM selectivities were low on this catalyst and they also decreased with increasing temperature. Thus, it appears that low temperatures favor high MF selectivities, making the high activity of RuOₓ domains particularly attractive as the CH₃OH oxidation component required for the synthesis of MF directly from methanol.

TABLE 3
Temperature effects on methanol oxidation on RuOₓ/ZrO₂
(4.2 wt Ru %; 4 kPa CH₃OH, 10 kPa O₂) at similar
CH₃OH conversions (˜22%; conversion is ˜10% at 353 k)

| Temperature | Conversion rate | Selectivity (%) | | | | |
|---|---|---|---|---|---|---|
| (K) | (mmol/g-Ru-h) | DME | HCHO | MF | DMM | COₓ |
| 353 | 34.4 | 0 | 9.7 | 82.2 | 5.6 | 2.6 |
| 373 | 78.1 | 0 | 8.0 | 78.3 | 6.3 | 7.5 |
| 393 | 174.5 | 0 | 6.5 | 69.6 | 5.5 | 18.5 |
| 408 | 279.2 | 0 | 5.8 | 64.9 | 4.0 | 25.3 |

Table 4 shows the effect of different temperatures on RuOₓ/Al₂O₃. While the reaction rates increased with increasing temperature from 393 K to 423 K, MF and HCHO selectivities increased and DMM selectivities concurrently decreased.

TABLE 4
Temperature effects on methanol oxidation on RuOₓ/Al₂O₃
(4.2 wt Ru %; 4 kPa CH₃OH, 10 kPa O₂) at similar
CH₃OH conversions (˜25%)

| Temperature | Conversion rate | Selectivity (%) | | | | |
|---|---|---|---|---|---|---|
| (K) | (mmol/g-Ru-h) | DME | HCHO | MF | DMM | COₓ |
| 393 | 147.9 | 0 | 13.5 | 34.4 | 50.1 | 2.0 |
| 408 | 215.1 | 0.3 | 16.8 | 38.9 | 39.7 | 4.3 |
| 423 | 312.8 | 0.6 | 31.1 | 41.6 | 20.9 | 6.6 |

Table 5 shows results obtained from oxidation of methanol at a temperature of 353 K (80° C.) using ruthenium oxide and oxides of two other platinum group metals, rhodium and palladium, all supported on titanium dioxide. It was noted that the supported palladium oxide catalysts were quite active and tended to produce an exothermic reaction.

TABLE 5
Methanol oxidation on TiO₂ supported RuOₓ, RhOₓ, and
PdOₓ (353 K, 4% CH₃OH, 9% O₂, ˜5% CH₃OH conversion)

| Catalyst | Conversion rate | Selectivity (%) | | | | |
|---|---|---|---|---|---|---|
| (metal wt %) | (mmol/g-metal-h) | DME | HCHO | MF | DMM | CO₂ |
| RuOₓ/TiO₂ | 17.0 | 0 | 22.8 | 70.5 | 6.7 | 0 |
| (4.1%) | | | | | | |
| RhOₓ/TiO₂ | 18.4 | 0 | 3.5 | 69.6 | 0 | 26.9 |
| (4.4%) | | | | | | |
| PdOₓ/TiO₂ | 21.6 | 0 | 13.5 | 85.4 | 0.4 | 0.8 |
| (4.4%) | | | | | | |

In another experiment, ethanol oxidation was studied on supported RuOₓ domains. Table 6 shows the reaction rates and selectivities at 393 K on RuOₓ/SiO₂ (4.3 wt % Ru) and RuOₓ/SnO₂ (4.1 wt % Ru). Acetaldehyde, diethoxyethane (acetal) and smaller amounts of ethyl acetate were formed. No diethyl ether and COₓ were detected at these low reaction temperatures. A trace amount of ethyl vinyl ether was always formed at these reaction conditions. RuOₓ domains on SnO₂ are more active than on SiO₂, as a result of more facile redox cycles for RuOₓ oxides on SnO₂, as was found for methanol oxidation. At low ethanol partial pressures (<4 kPa), the main product is acetaldehyde, with selectivities higher than 93%. The acetal (diethoxyethane, DEE) selectivities increased significantly from 0 to 81% as the ethanol pressures increased from 2 kPa to 40 kPa, concurrently with the decrease in the acetaldehyde selectivities from 97.3% to 17.6%. This is consistent with the reaction pathways of acetal formation, which involve the rate-determining step of acetaldehyde formation, and subsequent acetalization of acetaldehyde with ethanol. The ethanol conversion rates also increased with increasing the ethanol pressure in the range 2-40 kPa.

TABLE 6
Ethanol oxidation on RuOₓ/SiO₂ and RuOₓ/SnO₂ at 393 K (9% O₂, ˜10-15% C₂H₅OH conversion)

| | | Conversion | | | | | | |
|---|---|---|---|---|---|---|---|---|
| | Ethanol | rate | Selectivity (%) | | | | | |
| Catalyst | pressure | (mmol/g- | Diethyl | | Ethyl | Diethoxyethane | Ethyl vinyl | |
| (metal wt %) | (kPa) | Ru-h) | ether | Acetaldehyde | acetate | (acetal) | ether | COₓ |
| RuOₓ/SiO₂ (4.3%) | 4 | 59.9 | 0 | 97.1 | 0 | 2.9 | trace | 0 |
| RuOₓ/SnO₂ (4.1%) | 4 | 162.5 | 0 | 93.4 | 2.0 | 4.6 | trace | 0 |
| RuOₓ/SnO₂ (4.1%) | 2 | 115.8 | 0 | 97.3 | 2.7 | 0 | trace | 0 |
| RuOₓ/SnO₂ (4.1%) | 10 | 225.4 | 0 | 64.8 | 2.2 | 33.0 | trace | 0 |
| RuOₓ/SnO₂ (4.1%) | 20 | 277.6 | 0 | 30.8 | 1.2 | 68.0 | trace | 0 |
| RuOₓ/SnO₂ (4.1%) | 30 | 313.4 | 0 | 20.9 | 1.3 | 77.8 | trace | 0 |

Table 7 shows the results of oxidation of a mixed methanol/ethanol feed. Here, oxidation of a mixture that contained significant amounts of both compounds produced primarily acetals 1-methoxy-1-ethoxyethane and 1,1-diethoxyethane (DEE), along with acetaldehyde.

TABLE 7
Oxidation of CH₃OH + C₂H₅OH (1/4 molar ratio) on RuOₓ/SnO₂ at 393 K (4%
CH₃OH, 16% C₂H₅OH, 15% O₂, 10.2% C₂H₅OH conversion and ˜3.4% CH₃OH conversion).

| | Selectivity (%) | | | | | | | | |
|---|---|---|---|---|---|---|---|---|---|
| Conversion | | | | | 1-methoxy- | | | Ethyl | |
| rate | | | Methyl | Dimethoxy- | 1- | 1,1-diethoxyethane | Ethyl | vinyl | |
| (mmol/g-Ru-h) | HCHO | CH₃CHO | formate | methane | ethoxyethane | (acetal) | acetate | ether | CO₂ |
| 303.8 | 0 | 21.6 | trace | 1.5 | 34.7 | 42.4 | 0.5 | trace | 0.8 |
| (ethanol) | | | | | | | | | |
| 100.9 | | | | | | | | | |
| (methanol) | | | | | | | | | |

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A process for the oxidation of methanol, ethanol, or mixtures thereof comprising contacting the methanol and/or ethanol with an oxygen-containing gas and a supported catalyst comprising one or more platinum group metal oxides.

2. A process according to claim 1 comprising oxidation of methanol.

3. A process according to claim 2 in which the product of the process comprises primarily methyl formate.

4. A process according to claim 2 in which the product of the process comprises dimethoxymethane and/or formaldehyde.

5. A process according to claim 3 in which the product further comprises dimethoxymethane and/or formaldehyde.

6. A process according to claim 1 comprising oxidation of ethanol.

7. A process according to claim 6 in which the product of the process comprises primarily diethoxyethane.

8. A process according to claim 1 comprising oxidation of a mixture of methanol and ethanol.

9. A process according to claim 1 in which the surface density of the platinum group metal oxide or oxides on the support is from about 20% to about 300% of the surface density of a monolayer of said oxide or oxides.

10 A process according to claim 1 in which the surface density of the platinum group metal oxide or oxides is approximately that of a monolayer of oxide or oxides.

11. A process according to claim 1 in which the support comprises a material selected from alumina, silica, zirconia, titania, and mixtures thereof.

12. A process according to claim 11 in which the support comprises alumina.

13. A process according to claim 11 in which the support comprises silica.

14. A process according to claim 11 in which the support comprises zirconia.

15. A process according to claim 11 in which the support comprises titania.

16. A process according to claim 11 in which the support comprises stannic oxide.

17. A process according to claim 1 in which the support comprises one or more reducible metal oxides.

18. A process according to claim 17 in which the one or more reducible metal oxides are selected from reducible oxides of tin, iron, cerium, manganese, cobalt, nickel, chromium, zirconium, rhenium, titanium, silver and copper, and mixtures thereof.

19. A process according to claim 17 in which the one or more reducible metal oxides are selected from reducible oxides of tin, iron, cerium, zirconium, and mixtures thereof.

20. A process according to claim 17 in which the one or more reducible metal oxides comprises stannic oxide.

21. A process according to claim 17 in which the support comprises one or more layers of a reducible metal oxide or a mixture of such oxides disposed on a particulate alumina, silica, zirconia, or titania.

22. A process according to claim 21 in which the support comprises a layer of stannic oxide disposed on a particulate alumina, silica, titania, or zirconia.

23. A process according to claim 1 in which the catalyst comprises one or more ruthenium oxides.

24. A process according to claim 1 in which the catalyst comprises one or more rhodium oxides.

25. A process according to claim 1 in which the catalyst comprises one or more palladium oxides.

26. A process according to claim 1 in which the temperature is from about 30 to about 300° C.

27. A process according to claim 1 in which the temperature is from about 50 to about 180° C.

28. A process according to claim 1 in which the temperature is from about 80 to about 180° C.

29. A process for the production of a product comprising methyl formate comprising contacting a material comprising methanol with an oxygen-containing gas and a supported catalyst comprising one or more platinum group metal oxides.

30. A process according to claim 29 in which the product of the process further comprises dimethoxymethane and/or formaldehyde.

31. A process according to claim 29 in which the surface density of the platinum group metal oxide or oxides on the support is from about 20% to about 300% of the surface density of a monolayer of said platinum group metal oxide or oxides.

32 A process according to claim 27 in which the surface density of the platinum group metal oxide or oxides is approximately that of a monolayer of said oxide or oxides.

33. A process according to claim 29 in which the support comprises a material selected from alumina, silica, zirconia, titania, and mixtures thereof.

34. A process according to claim 33 in which the support comprises alumina.

35. A process according to claim 33 in which the support comprises silica.

36. A process according to claim 33 in which the support comprises zirconia.

37. A process according to claim 33 in which the support comprises titania.

38. A process according to claim 33 in which the support comprises stannic oxide.

39. A process according to claim 29 in which the support comprises one or more reducible metal oxides.

40. A process according to claim 39 in which the one or more reducible metal oxides are selected from reducible oxides of tin, iron, cerium, manganese, cobalt, nickel, chromium, zirconium, rhenium, titanium, silver and copper, and mixtures thereof.

41. A process according to claim 39 in which the one or more reducible metal oxides are selected from reducible oxides of tin, iron, cerium, zirconium, and mixtures thereof.

42. A process according to claim 39 in which the one or more reducible metal oxides comprise stannic oxide.

43. A process according to claim 39 in which the support comprises one or more layers of a reducible metal oxide or a mixture of such oxides disposed on a particulate alumina, silica, zirconia, or titania.

44. A process according to claim 43 in which the support comprises a layer of stannic oxide disposed on a particulate alumina, silica, titania, or zirconia.

45. A process according to claim 29 in which the catalyst comprises one or more ruthenium oxides.

46. A process according to claim 29 in which the catalyst comprises one or more rhodium oxides.

47. A process according to claim 29 in which the catalyst comprises one or more palladium oxides.

48. A process according to claim 29 in which the temperature is from about 30 to about 300° C.

49. A process according to claim 29 in which the temperature is from about 50 to about 180° C.

50. A process according to claim 29 in which the temperature is from about 80 to about 180° C.

51. A process for the production of a product comprising diethoxyethane comprising contacting a material comprising ethanol with an oxygen-containing gas and a supported catalyst comprising one or more platinum group metal oxides.

52. A catalyst comprising one or more platinum group metal oxides supported on a support comprising one or more layers comprised of a reducible metal oxide or a mixture of reducible metal oxides, the reducible oxide layer or layers being disposed on a particulate support comprising alumina, zirconia, silica, titania, zirconia, stannic oxide, or a mixture of two or more thereof.

53. A catalyst according to claim 52 in which the surface density of the ruthenium oxide on the support is greater than that for the monomeric isolated platinum group metal oxide or oxides.

54. A catalyst according to claim 52 in which the reducible metal oxide is selected from reducible oxides of tin, iron, cerium, manganese, cobalt, nickel, chromium, zirconium, rhenium, titanium, silver and copper, and mixtures thereof.

55. A catalyst according to claim 52 in which the reducible metal oxide is selected from reducible oxides of tin, iron, cerium, zirconium, and mixtures thereof.

56. A catalyst according to claim 52 in which the reducible metal oxide comprises stannic oxide.

57. A catalyst according to claim 52 comprising one or more ruthenium oxides.

58. A catalyst according to claim 52 comprising one or more rhodium oxides.

59. A catalyst according to claim 52 comprising one or more palladium oxides.

60. A catalyst according to claim 52 in which the surface density of the platinum group metal oxide or oxides on the support is from about 20% to about 300% of the surface density of a monolayer of said platinum group metal oxide or oxides.

61. A catalyst according to claim 60 comprising one or more ruthenium oxides.

62. A catalyst according to claim 60 comprising one or more rhodium oxides.

63. A catalyst according to claim 60 comprising one or more palladium oxides.

64. A catalyst according to claim 52 in which the surface density of the platinum group metal oxide or oxides on the support is approximately that of a monolayer of said oxide or oxides at the surface of the support.

65. A catalyst according to claim 64 comprising one or more ruthenium oxides.

66. A catalyst according to claim 64 comprising one or more rhodium oxides.

67 A catalyst according to claim 64 comprising one or more palladium oxides.
